(19) United States
(12) Patent Application Publication
(10) Pub. No.: US 2017/0071581 A1
(43) Pub. Date: Mar. 16, 2017

(54) INTEGRATED WIRELESS FERTILITY TRACKING SYSTEM

(71) Applicant: Kindara, Inc., Boulder, CO (US)

(72) Inventors: William Sacks, Boulder, CO (US); Katherine Bicknell, Boulder, CO (US)

(73) Assignee: Kindara, Inc., Boulder, CO (US)

(21) Appl. No.: 15/134,737

(22) Filed: Apr. 21, 2016

Related U.S. Application Data
(63) Continuation of application No. PCT/US2014/061797, filed on Oct. 22, 2014.
(60) Provisional application No. 61/894,098, filed on Oct. 22, 2013.

(51) Int. Cl. A61B 10/00; G01K 13/00; G01K 1/02

(52) U.S. Cl. CPC A61B 10/0012; G01K 1/02; G01K 13/002; A61B 2010/0019

(57) ABSTRACT

Described herein are various embodiments of an Integrated Wireless Fertility Tracking System. Various embodiments include a fertility thermometer and processes for collecting and charting fertility data for a female. Some embodiments have increased usability relative to conventional systems, making fertility charting easier and more effective for the user and thereby increasing user satisfaction and user compliance, which in turn increases effectiveness of the system.

### CROSS-REFERENCE TO RELATED APPLICATIONS

This application claims the benefit under 35 U.S.C. §120 as a continuation of International Application No. PCT/US2014/061797, filed in the U.S. Patent and Trademark Office as a Receiving Office on Oct. 22, 2014, which claims priority to U.S. Provisional Patent App. No. 61/894,098, titled “Integrated wireless fertility tracking system,” and filed on Oct. 22, 2013, the entire contents of both of which are hereby incorporated by reference.

### SUMMARY

In one embodiment, there is provided an apparatus comprising a thermometer. The thermometer comprises a wireless transceiver, at least one accelerometer, a temperature sensor, at least one processor, and at least one storage. The at least one storage has encoded thereon executable instructions that, when executed by the at least one processor, cause the at least one processor to carry out a method. The method comprises detecting, via the at least one accelerometer, that the thermometer has been picked up by a user and, in response to detecting that the thermometer has been picked up by the user, transmitting at least one message via the wireless transceiver to a computing device. The at least one message comprises an instruction to the computing device to cease outputting of an alarm.

In another embodiment, there is provided an apparatus comprising a thermometer. The thermometer comprises a wireless transceiver, at least one accelerometer, a temperature sensor, a display screen comprising a light source, at least one processor, and at least one storage. The at least one storage has encoded thereon executable instructions that, when executed by the at least one processor, cause the at least one processor to carry out a method. The method comprises detecting, via the at least one accelerometer, that the thermometer has been picked up by a user and, in response to detecting that the thermometer has been picked up by the user, illuminating the display screen with the light source.

In a further embodiment, there is provided an apparatus comprising a thermometer and a case. The thermometer comprises a first housing, a protrusion, and a temperature sensor. The case comprises an inner cavity disposed within the case, wherein at least a portion of the inner cavity comprises a shape corresponding to a shape of the protrusion of the thermometer. The case further comprises a switch and a disinfector, the disinfector comprising an ultraviolet-light-emitting circuit positioned to illuminate at least a portion of the inner cavity with ultraviolet light in response to a change in state of the switch. The thermometer and case are arranged such that at least a portion of the thermometer is insertable into the case and the switch is positioned such that the first housing of the thermometer contacts the switch when the portion of the thermometer is inserted into the case and changes state in response to the contact.

In another embodiment, there is provided an apparatus comprising a thermometer. The thermometer comprises a first housing, a protrusion from the first housing, a cap located on a distal end of the protrusion, and a temperature sensor. The cap comprises an exterior surface and the exterior surface comprises a conductive material. The cap is shaped such that a first dimension of the cap on a first axis perpendicular to a longitudinal axis of the protrusion is less than a second dimension of the cap on a second axis perpendicular to the longitudinal axis.

In a further embodiment, there is provided an apparatus comprising a thermometer. The thermometer comprises a first housing, a protrusion from the first housing, and a temperature sensor. The protrusion has a consistent cross-sectional width for a majority of a length of the protrusion, and the consistent cross-sectional width has a value in a range of 0.10-0.15 inches.

In another embodiment, there is provided an apparatus comprising a thermometer and a case. The thermometer comprises a protrusion, a cap located on a distal end of the protrusion, and a temperature sensor. The cap comprises an exterior surface and the exterior surface comprises a conductive material. The cap is shaped such that a first dimension of the cap on a first axis perpendicular to a first longitudinal axis of the protrusion is less than a second dimension of the cap on a second axis perpendicular to the first longitudinal axis. The case comprises a second housing having a second exterior surface and an inner cavity disposed within the case. At least a portion of the inner cavity comprises a shape corresponding to a shape of the protrusion of the thermometer. The shape of the cavity has a third dimension on a third axis perpendicular to a second longitudinal axis of the cavity that is less than a fourth dimension of the cavity on a fourth axis perpendicular to the second longitudinal axis. The thermometer and case are arranged such that at least a portion of the thermometer is insertable into the case. The first dimension is larger than the third dimension and the third dimension is less than the fourth dimension.

In a further embodiment, there is provided an apparatus comprising a thermometer and a case. The thermometer comprises a first housing having a first exterior surface and the first exterior surface comprises a material. The thermometer further comprises a protrusion, a temperature sensor, and a display screen disposed within the first housing such that the material covers the display screen. The case comprises a second housing having a second exterior surface, and an inner cavity disposed within the case. At least a portion of the inner cavity comprises a shape corresponding to a shape of the protrusion of the thermometer. The thermometer and case are arranged such that at least a portion of the thermometer is insertable into the case. The first exterior surface and second exterior surface are shaped such that, when the thermometer is inserted into the case, the first exterior surface and second exterior surface form a unified shape. The unified shape has a continuous form at an intersection of the thermometer and the case.

In another embodiment, there is provided an apparatus comprising a thermometer. The thermometer comprises a first housing, a protrusion from the first housing that comprises a conductive material disposed on a distal end of the protrusion, and a temperature sensor. The thermometer is arranged for use as an oral thermometer in which the distal end will be located underneath a user's tongue. The protrusion comprises a teeth-gripping region, the teeth-gripping region being a section of the protrusion to be gripped by a user's teeth during use. The teeth-gripping region is located on the protrusion at a distance from the distal end of the protrusion that corresponds to a distance between front teeth and a rear region of an underside of a tongue of an average adult human. A center of gravity of the thermometer is located between 0.5 and 1 inches from the teeth-gripping region of the protrusion.

In a further embodiment, there is provided an apparatus comprising a thermometer and a case. The thermometer comprises a first housing, a first electrical contact disposed on an exterior of the first housing, a protrusion, a first battery, and a temperature sensor. The case comprises a second housing, a second electrical contact disposed on an exterior of the second housing, and an inner cavity disposed within the case. At least a portion of the inner cavity comprises a shape corresponding to a shape of the protrusion of the thermometer. The case further comprises a second battery, an external port via which to receive power from an external source, and a charging circuit electrically connected to the external port, to the second battery, and to the second electrical contact. The thermometer and case are arranged such that at least a portion of the thermometer is insertable into the case, and the first electrical contact and second electrical contact are positioned such that they contact one another when the portion of the thermometer is inserted into the case. The charging circuit is configured to selectively charge the first battery and/or the second battery using power received via the external port. The charging circuit is configured to select at a time whether to charge the first battery and/or the second battery based at least in part on whether the thermometer is inserted into the case and on a current charge of the first battery and/or the second battery.

In another embodiment, there is provided an apparatus comprising a thermometer. The thermometer comprises a temperature sensor, at least one processor, and at least one storage having encoded thereon executable instructions that, when executed by the at least one processor, cause the at least one processor to carry out a method. The method comprises, following a start of reading of a temperature with the temperature sensor, detecting a completion of the reading of the temperature and, in response to detecting the completion, providing a signal to a user that the reading is complete, wherein providing the signal comprises providing a haptic and/or visual signal to the user.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a sketch of an exemplary pair of a computing device and a thermometer and case with which some embodiments may operate;

FIGS. 2A, 2B, and 2C illustrate exemplary embodiments of a thermometer and case, and exemplary components thereof;

FIG. 3A is a sketch of an example of a thermometer in accordance with some other designs, with which some embodiments may operate;

FIG. 3B is a sketch of an example of a thermometer in accordance with some embodiments;

FIG. 4A is a sketch of an example of a thermometer probe and tip in accordance with some other designs, with which some embodiments may operate;

FIG. 4B is a sketch of an example of a thermometer probe and tip in accordance with some embodiments;

FIG. 5A shows steps of several example processes, with which some embodiments may operate; and

FIG. 5B shows an example of steps that may be included in a process that may be implemented in some embodiments.

### DETAILED DESCRIPTION

Charting oral basal body temperature (BBT) and other fertility signs is an effective way to determine the fertility status of human females throughout the menstrual cycle. Many women undertake fertility charting as an aid to conception, as a way to effectively avoid pregnancy, as a way to better understand their gynecological health, or as a way to identify and characterize fertility problems. Conventional methods rely on simple manual thermometers with generally the same design as fever thermometers, and paper charts.

The inventors have recognized and appreciated that, although the conventional methods depend on consistent use to produce dependable results, the conventional methods suffer from usability problems that often reduce consistent use over time. Because women typically chart their fertility over a period of multiple months, the usability problems result in poor data and poor results when the conventional methods are used.

Described herein are various embodiments of an Integrated Wireless Fertility Tracking System. Various embodiments include a fertility thermometer and processes for collecting and charting fertility data. Some embodiments have increased usability relative to conventional systems, making fertility charting easier and more effective for the user and thereby increasing user satisfaction and user compliance, which in turn increases effectiveness of the system. Techniques herein may, in some embodiments, be used together with techniques and apparatuses described in U.S. patent application Ser. No. 13/696,438, filed on Nov. 6, 2012, and titled “System for tracking female fertility” (“the '438 application”), and/or in any of the applications to which the '438 application claims priority, including U.S. Patent Applications Ser. Nos. 61/332,701, filed May 7, 2010, 61/350,084, filed Jun. 1, 2010, and 61/354,182, filed Jun. 11, 2010, and International Patent Application Serial No. PCT/US2011/027196, filed Mar. 4, 2011 (“the Priority Applications”). The '438 application and all of the Priority Applications are incorporated herein by reference in their entireties and at least for their discussion of techniques and apparatuses for tracking female fertility.

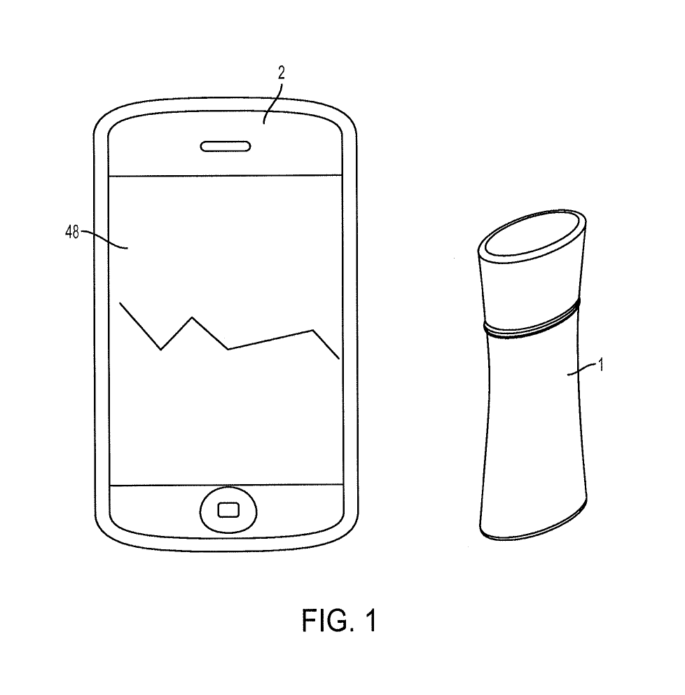

One embodiment of an Integrated Wireless Fertility Tracking System is illustrated in FIG. 1. As shown in FIG. 1, this embodiment includes an oral fertility device (1) to collect oral temperature readings from a user, which may be a female, including a human female. The oral fertility device (1) is arranged to wirelessly transmit the oral temperature readings—in some cases along with the time and date of each reading—to another device. In the embodiment of FIG. 1, this other device is an Internet-enabled device (2), which may be any suitable computing device, for example a smartphone. The device (2) may be configured to execute a software application (48). The software application (48) may carry out various functions relating to fertility data for the user. Such fertility data may include oral temperature readings received from the oral fertility device (1). The software application (48) may, for example, record/store fertility data for the user in one or more storages of the device (2), display the fertility data via a user interface of the device (2), analyze/process the fertility data in some manner, and share the fertility data by transmitting the fertility data via one or more wired and/or wireless computer networks to one or more other devices.

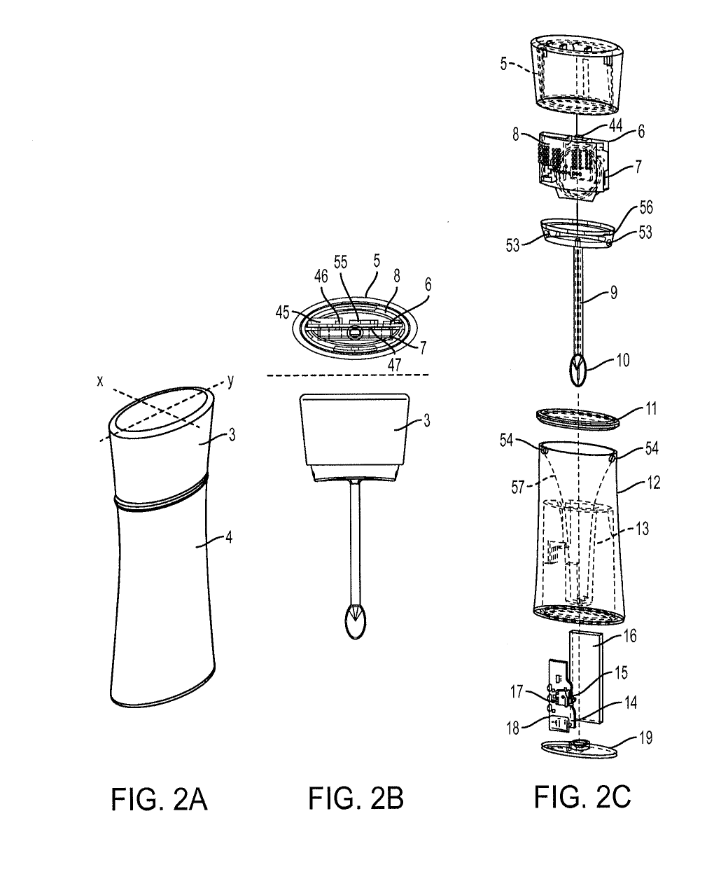

FIGS. 2A-2C illustrate additional details of one embodiment of the oral fertility device (1) of FIG. 1. As shown in FIGS. 2A-2C, the fertility device is composed of an integrated oral thermometer (3) and case (4). The thermometer (3) includes a thermometer head casing (5), a battery (7), circuit board (6) containing a microprocessor, temperature circuit, accelerometer, wireless transceiver, and screen driver, a flexible screen (8), a probe (9) and probe tip (10). The probe tip (10) may be formed of a conductive material, which may be a metal. The thermometer case (4) includes a trim ring (11), molded case body (12) that includes an internal guide (13) for the probe (9) of the thermometer (3), UV disinfecting light (14), position-sensing switch (15), battery (16) and control circuit (17) that manages the charge of the battery and activates the UV disinfecting light (14) when the thermometer is placed back in the base, and charging port (18). The case (4) also includes a base plate (19).

Embodiments may include/implement one or more, in any combination, of the following functionalities.

First Functionality

With reference to FIGS. 2A-2C, the thermometer (3) and case (4), when fitted together such that the probe (9) and probe tip (10) are inside the body (12) and internal guide (13), may form an integrated shape that is not suggestive of a thermometer. Additionally or alternatively, the screen (8) may be positioned on the thermometer (3) such that the screen (8) is concealed behind a translucent surface material of the head casing (5), making the screen (8) difficult to see, or invisible, when the screen (8) is not illuminated. These two features, alone or in combination, may prevent the device (1) from being easily identified as a fertility tracking device or as a medical device.

This may be advantageous for some women who are charting their fertility to achieve or avoid pregnancy. Thermometers using other designs are easily identifiable as such and therefore easily communicate to others that a user may be sick, or possibly that the user is charting fertility. As family planning may be a sensitive topic for some users, home users of thermometers having other designs may go to lengths to place their thermometers out of sight after use. Similarly, for women who take thermometers having other designs with them to various locations (their partner's house, while traveling, etc.), these other thermometers may also communicate sickness and/or fertility tracking if seen being carried or in a purse and such women take steps to conceal them.

Embodiments that incorporate this shape may remove or mitigate this concern by concealing or obscuring the nature and function of the device (1) when it is not in operation. Embodiments that incorporate this shape may therefore allow users to chart fertility without overtly communicating this fact to those around them. Some users may therefore feel more comfortable leaving the device on a bedside table or otherwise in the open at home, or not feel nervous or embarrassed traveling with the device. For those for whom fertility tracking is a sensitive subject, this makes the process of fertility charting easier and more convenient for the user. Additionally the usability of the process is improved since the thermometer is more likely to be close at hand instead of hidden out of sight.

In some embodiments that incorporate this shape, the device (1) may be formed in part by molding the thermometer head casing and case body from identical semi-translucent material (e.g., plastic, rubber, latex, urethane, or any another suitable material, as embodiments are not limited in this respect) and by modeling the thermometer and case as one object with a unified shape as shown in FIG. 2A. By forming the device in this manner, the transition between the head and the case may be slight. Such a slight transition may mean that, for example, a shape of the exterior of the case (4) at the interface of the case (4) and the thermometer (3) may match a shape of the exterior of the thermometer (3). The shapes may match when there is a continuity in a shape that is formed when the shapes of the respective parts are positioned adjacent to one another. Such a continuity may be a continuity that is perceptible to a casual observer such that the casual observer will interpret the thermometer and case as one unified shape. The shape of the entire object may be very different from existing thermometers, and may be considered to be more similar to cosmetic products. The object therefore may not communicate to a casual observer that it includes a thermometer.

To hide/obscure the screen when not illuminated, the screen (8) may be positioned on a flexible circuit board that fits within the shape of the inside of the thermometer head case (5). When illuminated and in operation, the screen (8) shines through the thermometer head material. When it is not illuminated, the screen (8) is obscured by the thermometer head material and thereby invisible or obscured. This may enable the thermometer to masquerade as some other type of object. To power the screen (8), the screen (8) is attached via a flexible connector to a control circuit board (6) and battery (7), which may also be placed inside the head case.

Second Functionality

With reference again to FIGS. 2A-2C, in some embodiments, the device (1) may include an integrated Ultra-Violet disinfector within the case (4). The UV disinfector may act to disinfect (which may include destroying some, most, more than 90 percent of, nearly all, or a medically-significant amount of bacteria) the thermometer probe (9) and tip (10) between uses, when the probe (9) and tip (10) are inserted into the case (4). Thermometers of other designs do not include a disinfector, and if disinfecting is desired (as is recommended), the thermometers must be separately cleaned with soap and water. Embodiments that include the UV disinfector enable the probe and tip to be disinfected between uses. For germ-conscious users, this may make the device (1) easier to use and more convenient than other thermometers, which may increase compliance for these users.

The UV disinfector consists of a battery (16), control circuit (17), charging port (18), UV LED or light bulb (14), and a sensing switch (15) mounted in the thermometer base. The control circuit (17) may include a processor executing instructions to carry out a process for controlling the UV disinfector.

In some embodiments, the switch (15) may be a switch that is operated by a user to operate the disinfector. In other embodiments, the probe (9) and switch (15) may be positioned such that, when the thermometer (3) is inserted into the case (4) after use, the probe (9) changes a state of the switch (15) without the user needing to take a separate action to operate the switch. The switch (15) may be implemented in any suitable manner, as embodiments are not limited in this respect. In some embodiments, the switch (15) may be a position sensing displacement switch that is tripped by the probe tip (10) or probe base (56), an optical position sensing switch that registers the presence of the probe (9) or probe tip (10) inside the probe guide (13), an electrical sensing switch that uses electrical connections on the thermometer head (53) to detect that the thermometer has been replaced in the case, or any other kind of switch.

In some embodiments, in response to detecting a change in state of the switch (15), the UV disinfector circuit (17) turns on the light (14) to disinfect the probe (9) and/or probe tip (10) for a time, then turns the light (14) off. The time may be any suitable period of time, as embodiments are not limited in this respect. In some embodiments, the time may be a set interval programmed into a storage of the UV disinfecting circuit (17).

In some embodiments, for the UV disinfecting light shine on the probe, the probe guide (13) includes a transparent window. This window may be molded in transparent plastic as part of the probe guide (13). When the thermometer (3) is placed back in the case (4) and the disinfecting circuit (17) activates the UV light (14), light shines through the window in the probe guide (13) and disinfects the probe. The window may enable the probe (9) and tip (10) to be disinfected while keeping the probe (9) and tip (10) physically separate from the UV light (14) and electronics (15-18) of the UV disinfector.

The UV disinfector battery (16) charges through a charging port (18) (which may be any suitable port, including a mini or micro USB) mounted in the thermometer case (4). In embodiments in which the charging port (18) is located on or near the bottom of the case (4), the base plate (19) may have an opening through which a charging cable may attach.

Third Functionality

In embodiments, as shown in FIGS. 2A-2C, the device (1) may charge both the battery (7) in the thermometer (3), which powers the control circuit, transceiver, screen and thermometer circuitry, and the battery (16) in the case (4), which powers the UV disinfector, using the same case-mounted charging port (18). This may be accomplished by adding a discrete electrical connection between the thermometer head (53) and the case (54).

Additionally, in some embodiments, a programmed microcontroller (17) in the case (4) and a programmed microcontroller (55) in the thermometer (3) may manage the charge of the respective batteries to route charging energy to the batteries as appropriate to ensure both batteries are maximally charged. This may enable the device to have a single charging port, which may be advantageously hidden when the device (1) is in some positions (e.g., standing up) to conceal the fact that the device (1) is an electronic object.

The balancing of charging of the two batteries may be accomplished by the microcontroller (17) in the base sensing, through an electrical connection, the current charge of each battery. The microcontroller (17) may execute instructions and, in accordance with a programmed sequence of operations defined by the instructions, and known techniques to route charging energy first to the battery (7) in the thermometer (3) until it is fully charged as determined by its voltage, and then routing charging energy to the battery (16) in the case (4) until it is fully charged. Additionally, the microcontroller (17) in the base may prioritize charging the battery (7) in the thermometer (3) when the device isn't plugged in, and therefore send it charging energy to make sure the battery (7) in the thermometer (3) has sufficient charge to operate even when the battery (16) in the case (4) is fully drained.

In some embodiments, the battery (7) in the thermometer (3) may be smaller than the battery (16) in the case (4). For example, the battery (7) may be physically smaller and hold a lesser charge than the battery (16). In such embodiments, using a battery (7) that is physically smaller may aid in reducing a size of the thermometer (3) and/or case (4), which may be advantageous in some embodiments. Further, through the charge-balancing functionality discussed in this section, in some such embodiments the thermometer (3) may not have a limited battery lifespan despite having a battery (7) that is physically smaller and holds a lesser charge.

It should be appreciated that, in some embodiments, the device (1) may include two charging ports, one on the thermometer (3) and one on the case (4), as embodiments are not limited in this respect.

Fourth Functionality

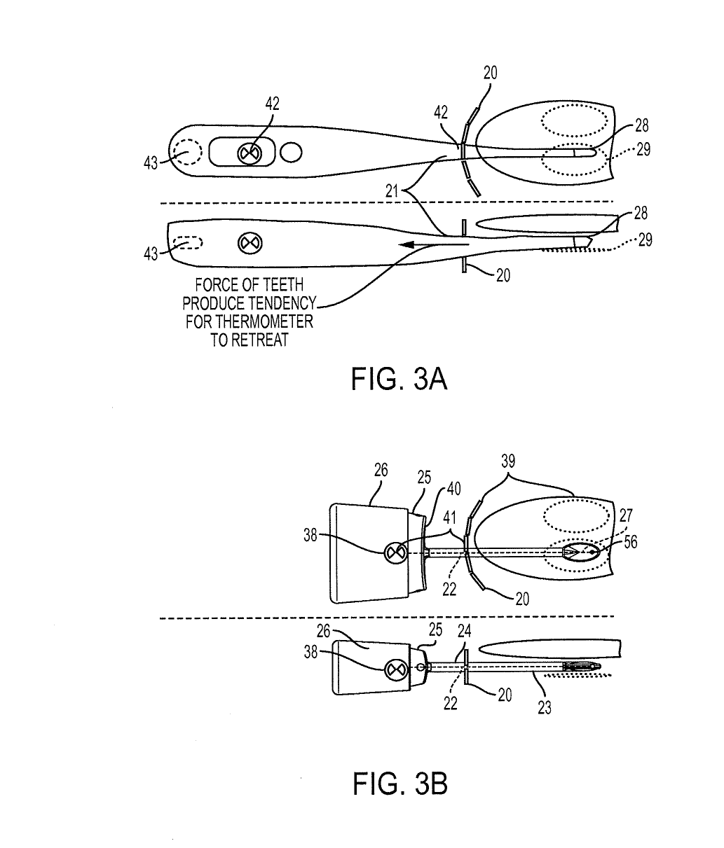

As shown in FIG. 3B (and with reference to FIGS. 2A-2C), in some embodiments the oral thermometer probe (9) may not have a taper, but instead may have a consistent cross-sectional width between the interface (40) and the probe tip (10). Such a consistent cross-sectional width may be the cross-sectional width for the entirety of the probe (9) between the interface (40) and tip (10) or for a majority of the probe (9). For example, in some embodiments, the probe (9) may have a consistent cross-sectional width for more than half, or more than two-thirds, or more than 90 percent of the length of the probe (9) between the interface (40) and tip (10). In some embodiments, the probe (9) may have a circular shape in cross section, and the consistent cross-sectional width may be a diameter. In some such embodiments, the probe (9) may have a constant diameter of a size (e.g., in a range of 0.10-0.15 inches, including a diameter of 0.115 inches) that, for most humans, fits between and can be easily grasped by the user's teeth (20) for comfort while taking a temperature.

Thermometers implementing a different design, such as the one illustrated in FIG. 3A, use a tapered probe design (21) that acts like a wedge in a user's mouth and makes it difficult to hold the thermometer still, as the natural motion of an inclined plane with forces applied to its faces is to retreat in the direction opposite to its point.

In these embodiments, the probe (9), in contrast, may not have a natural proclivity to retreat (in a direction along a longitudinal axis of the probe (9)) from a user's mouth when grasped by the teeth or lips, or may have a reduced natural proclivity as compared to other thermometers that include the taper. Additionally, in some such embodiments, the probe (9) may have a diameter (22) that enables, for most humans, the probe (9) to be conveniently grasped between individual teeth. Gripping the probe (9) between individual teeth may more comfortable for most users than holding it with lips (as users often do with other thermometers), since the user may be able to close or mostly close her jaw (her top and bottom teeth reaching a proximity of 0.10-0.15 inches in the closed position) and mouth and relax her face and lip muscles while taking the measurement. These features may make our system more comfortable for the user (which increases compliance), and reduces the likelihood of movement during the measurement (thus increasing accuracy of measurement).

To achieve this non-tapered shape, in some embodiments the probe (9) may be molded from a flexible material (e.g., rubber, silicone, plastic, or other material, as embodiments are not limited in this respect). In one embodiment, the lower half (23) of the probe (9) may be more flexible than the upper half (24). This may enable the probe (9) to be more flexible where flexibility may enable greater comfort for users (e.g., where the lower half (23) may be located under the tongue), but allow for greater rigidity between the teeth and the thermometer head. Rigidity on the upper half (24) may reduce a likelihood that the thermometer head will hang downward from the mouth at an angle that is difficult for the user to read while the lower half (23) is inside the user's mouth.

In one embodiment, the probe (9) may include a smaller inner diameter (while keeping the outer diameter of the entire probe constant) closer to the top (24) to allow for extra rigidity and a secure connection with the thermometer head. The probe base (25) is large and fits into the thermometer head (26), which may allow for a secure and strong connection between the probe assembly and the thermometer head.

To assemble the thermometer (3), in some embodiments, the temperature sensor (56) is inserted into the probe tip (10), and then the leads from the sensor (27) are connected through the probe (9) from the bottom to the top before the probe tip assembly is securely fastened to the probe. The sensor leads are then attached to the circuit board inside the thermometer head (26).

Fifth Functionality

As shown in FIG. 3B (and with reference to FIGS. 2A-2C), in some embodiments, the thermometer (3) has a center of gravity (38) of the thermometer (3) close to the probe tip (10) to create a low amount of torque on the user's tongue, lips and teeth while the probe tip (10) is inside the user's mouth and a measurement is being taken. To achieve this, the battery, circuit board, and wireless transmitter are located close to the base of the probe. Furthermore, in some embodiments, the intersecting surface (40) between the probe (9) and thermometer head (26) is curved to accommodate the lips. This may enable the user to place the thermometer close to her mouth, with her lips contacting the surface (40), further reducing the torque the thermometer head (25) exerts on the users lips, mouth and teeth when cantilevered out of the mouth.

In some embodiments, the center of gravity (38) is (i) between 0.25 and 1 inches, and in some embodiments 0.5 inches, from the location (41) at which a user will place her teeth, and/or (ii) between 2.2 and 3.0 inches from the probe tip (10). This is in contrast to the thermometer shown in FIG. 3A, which may place the center of gravity between 1.5 and 2.5 inches from the teeth (42). The thermometer of FIG. 3A, by implementing such a design, impose a torque lever between 1.5 and 5 times greater than the torque lever that is imposed on a user's mouth by embodiments that place the center of gravity between 0.5 and 1 inches from the user's teeth.

In the embodiment of FIG. 3B, to locate the center of gravity close to the teeth, various components of the thermometer (3) are located close to the teeth (41). In some embodiments, the screen (8), circuit board (6), and battery (7) may be layered on top of each other and located in the head (26) closer to the probe base (25) than to the other side of the head (26). This is in contrast to the thermometer shown in FIG. 3A, which places the battery (43) at the far end of the thermometer past the screen, thus moving the center of gravity away from the teeth and decreasing user comfort.

Sixth Functionality

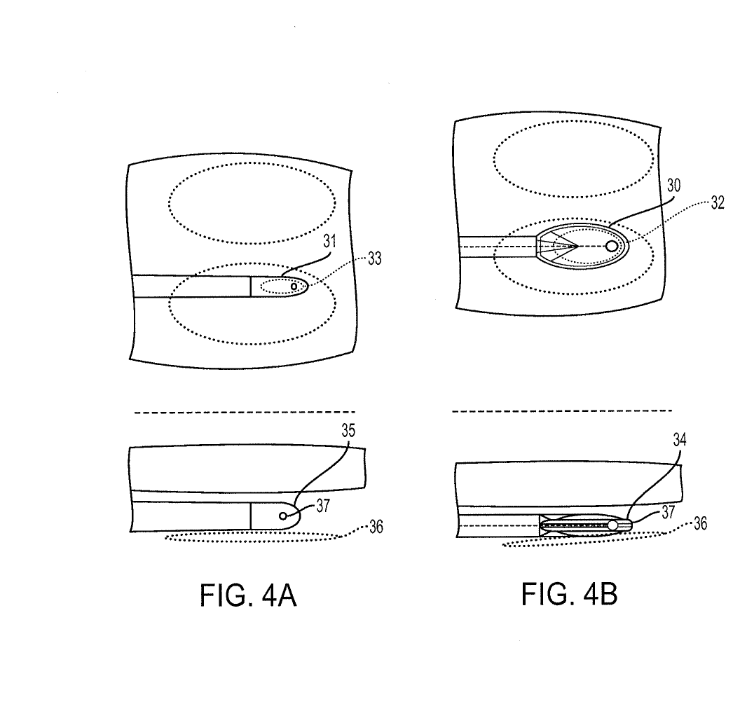

As shown in FIGS. 2A-2C and 3B, in some embodiments the oral thermometer probe tip (10) has a shape that enables heat transfer at a rate faster than may be achieved with tips of other shapes. Faster heat transfer may reduce the time needed for the thermometer (3) to take a measurement, as compared to a thermometer having the design illustrated in FIG. 4A, and may increase user comfort by decreasing the amount of time a user needs to hold the thermometer in her mouth. In other thermometers, probe tips are cylindrical with a pointed tip (28). This both makes the tip pointy and uncomfortable to rest against the back of the user's “heat pocket” (29) (e.g., an area underneath the tongue in which basal body temperature may be read), and slows the heat transfer from the users heat pocket to the temperature sensor inside the tip.

As shown in FIG. 4B, in some embodiments, the probe tip (10) achieves faster heat transfer to the temperature sensor in one or both of two ways.

First, in some embodiments, when viewed in cross-section, the probe tip (10) may be wider in one dimension than another. For example, a width (30) of the probe tip (10) when viewed from above (in “plan view”) may be larger than a height (34) of the probe tip (10) when viewed from the side (in “side view”). The width may be, for example, between two to three times larger than the height. The width (30) may be the dimension of the probe tip (10) in one axis perpendicular to a longitudinal axis of the probe (9) and the height (34) of the probe tip (10) may be the dimension of the probe tip (10) in another axis perpendicular to the longitudinal axis of the probe (9). As shown in FIG. 4B, the probe tip (10) may have a “disk” shape in some embodiments. Accordingly, the probe tip (10) may be flat when viewed from the side, or flatter in side view than a cylindrical probe tip (31) (35) of the thermometer design shown in FIG. 4A. Therefore, the probe tip (10) may have a larger area of contact equal to 0.07-0.09 square inches with a user's heat pocket (32) as compared with a probe tip (33) of other thermometer designs, which are in the range of 0.04-0.06 square inches. This larger area of heat transfer means the probe tip may heat faster to the temperature of the user's heat pocket.

Second, the probe tip (10) is flatter in side view (34) than a probe tip (35) of the thermometer design shown in FIG. 4A. This means that the distance from the heat pocket (36) to the temperature sensor (37) in the embodiment of FIG. 4B may be shorter than in the design illustrated in FIG. 4A. For example, the distance from the heat pocket (34) to the sensor (37) in the embodiment of FIG. 4B may be up to 4× shorter in some embodiments than in the thermometer of FIG. 4A. This results in faster heat transfer to the temperature sensor and a faster temperature measurement, which may make the thermometer more convenient for the user.

Accordingly, in some embodiments the probe tip (10) is flat in profile and rounded in plan view, which may match the shape of the user's heat pocket. This makes it more comfortable. Also, because there may be more metal in direct contact with the heat pocket, and a shorter distance from the surface of the probe tip to the temperature sensor, the thermometer of the embodiments of FIG. 4B accomplishes a faster heat transfer than with the thermometer of FIG. 4A, which may translate into a faster and more accurate measurement.

This shape of the probe tip (10) may be achieved, in some embodiments, by creating a custom die and stamping the probe tips from stainless steel. The temperature sensor may then be potted inside the probe tip with conductive epoxy and allowed to dry before the leads are fed through the probe, and the tip is attached to the probe with medical-grade adhesive. With some manufacturing processes, stamping the tip may enable achieving a desired shape while keeping the wall thickness low or at a minimum, which is advantageous for fast heat transfer.

Seventh Functionality

With reference to FIG. 2C, in some embodiments, the thermometer (3) is configured to provide haptic and/or visual feedback to the user in response to finishing taking a temperature measurement. Thermometers of other designs may be silent or provide an audible signal (e.g., a “beep”) to indicate the measurement has finished. Audible signals can be disadvantageous in the context of fertility charting. It is recommended that temperature be taken early in the morning before getting out of bed, and an audible signal can unintentionally wake up the user's partner in the morning. This may anger the user's partner and/or the user, and may reduce compliance. In some embodiments, rather than an audible feedback, the thermometer (3) may be configured to provide a haptic and/or visual signal. Haptic and/or visual signals may be less likely to disturb the partner of the user, who may be sleeping in the same bed as the user. Though, it should be appreciated that some embodiments may include an auditory signal. For example, in some embodiments, the thermometer may be configurable either to produce or not produce one or more of an audible signal, a haptic signal, and/or a visual signal in response to finishing a temperature measurement.

In embodiments that provide visual feedback, the visual feedback may be provided in any suitable manner. For example, in some such embodiments, the visual feedback may be presented via the screen (8). For example, visual feedback may be accomplished by the circuit board (6) and/or circuit board (17), executing instructions with a processor, detecting completion of a measurement and responding to the completion by displaying a graphic on the screen (8), such status bar, or by increasing the brightness of the screen (8).

In embodiments that provide haptic feedback, the haptic feedback may be provided in any suitable manner. For example, in some such embodiments, the haptic feedback may be accomplished with a haptic feedback chip on the circuit board (6). Such a haptic feedback chip may incorporate a linear resonant actuator, or may vibrate through any other suitable hardware or other technique. In such embodiments, the circuit board (6), executing instructions with a processor, may detect completion of a measurement and respond to the completion by controlling the haptic feedback chip to provide a haptic feedback pattern to the user, to indicate to the user that the measurement has finished. This enables the user to keep her eyes closed during the measurement and “feel” when it is finished through her teeth and lips or fingertips. This may allow her to remain in a more restful state during the measurement.

Eighth Functionality

With reference to FIG. 2C, in some embodiments, the thermometer (3) may include a wireless transceiver (e.g., a Bluetooth Low Energy (BLE)) transceiver (45) and/or an accelerometer (46). In these embodiments, the user may set an alarm (e.g., a wake-up alarm) on her smartphone (2) or other wireless device. The user may then be able to turn off the alarm, after the alarm goes off, simply by picking up and/or pushing a button (44) on the thermometer (3). This may make fertility charting easier, since the user can set her alarm on her smartphone or other device like she is accustomed (research has shown that a high percentage of women with smartphones use their smartphone as their alarm clock), yet turn off her alarm without touching her phone. As fertility charting techniques recommend charting fertility early in the morning, before getting out of bed, a user of the fertility tracking system may be woken by the alarm on a smartphone (or other device) and, as a first action following being awoken, pick up the thermometer (3) to take a temperature measurement. By configuring the thermometer (3) to communicate to the smartphone/device turn off the alarm when the thermometer (3) is picked up and/or when a button is pressed, the thermometer (3) can simplify the user's morning routine by removing the need to separately operate the smartphone/device to turn off the alarm.

In embodiments, this feature effectively turns the thermometer into an “off” button for the alarm on the smartphone (or other device). By turning the thermometer into the alarm “off” button (by means of an accelerometer or button) the user is able to accomplish two steps in one: turn off her alarm and pick up her thermometer to take her morning waking temperature. This feature may make fertility charting easier. The feature may also increase compliance by turning the thermometer into a haptic reminder to the user to take her temperature as soon as she wakes up and turns off her alarm, as her thermometer will already be in her hand when she turns off her alarm.

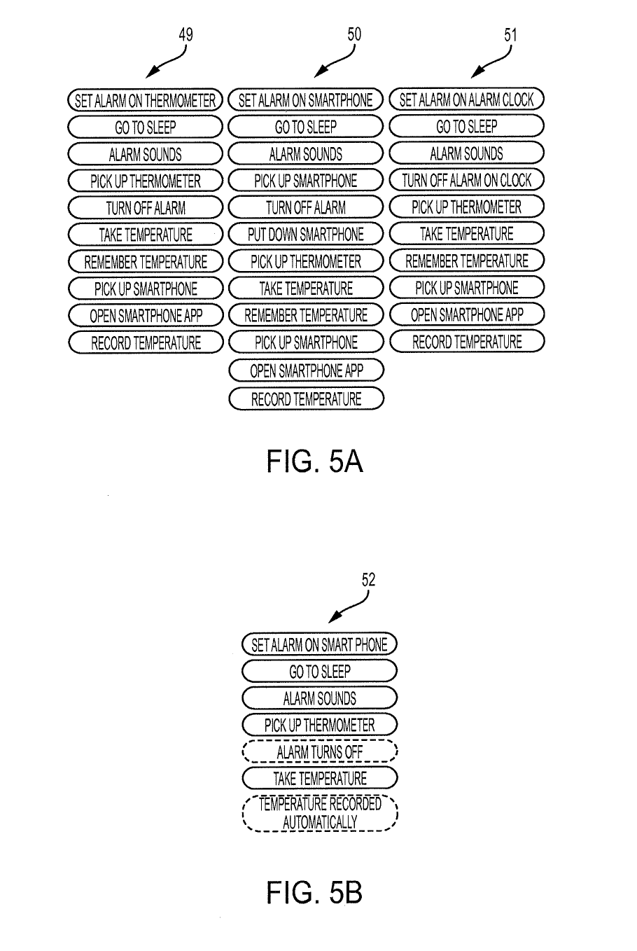

Embodiments may implement this feature in any suitable manner, as embodiments are not limited to implementing any particular process. FIG. 5B illustrates one example of a process by which the thermometer (3) may be operated as an alarm “off” button for another device.

FIG. 5A also illustrates other processes that may be implemented by thermometers of other designs. As shown in FIG. 5B, processes that may be implemented in some embodiments may be advantageous as compared to the user workflow with current fertility thermometers that include either integrated alarms or no alarms. Integrated alarms may be disadvantageous in that they are operated via the user interface of the thermometer, which may have a small screen and buttons that may be difficult to use, and also must be set when the user is holding the thermometer. In embodiments, the process implemented by the thermometer (3) in communication with a smartphone or other device may fit a user's current or pre-existing workflow. This may be because the user may traditionally have set an alarm on her phone/device, as discussed above, and using the thermometer (3) in connection with that phone/device may not be a change in routine that would qualify as disconcerting or as a difficult adjustment for some users.

As shown in FIG. 5A, other processes for waking up and taking basal body temperature have between 10 and 12 steps (49,50,51). In contrast, processes that may be carried out using some embodiments (e.g., the process of FIG. 5B) that incorporate this feature have five steps. This may increase ease of use and convenience for users, thereby increasing compliance and improving results.

With reference again to FIG. 2C, in some embodiments, this functionality may be implemented by a processor of the circuit board (6) and/or circuit board (17) executing instructions stored in one or more storages. The instructions may embody a process including various acts.

In some embodiments, the process may include initializing communication between the thermometer (3) and the smartphone (or other device). The initializing of communication may be carried out in any suitable manner, as embodiments are not limited in this respect. For example, in some embodiments that implement the Bluetooth® protocol, the thermometer (3) may be “paired” with the smartphone (or other device) using a conventional Bluetooth pairing process. As such pairing processes are known, they will not be discussed further herein. In other embodiments, however, such as embodiments that include a BLE transceiver, a pairing process may not be carried out during initialization. In embodiments that include an initialization, following initialization the thermometer (3) may communicate with the smartphone/device.

In some embodiments, the process may also include detecting a user operation that signals that an alarm should be turned off. The user operation may be any suitable operation, as embodiments are not limited in this respect. For example, the process may include detecting, via a signal received from one or more accelerometers, that the user has picked up the thermometer (3). As another example, the process may include additionally or alternatively detecting a change in state of a mechanical or electrical switch on the exterior of the thermometer head (53), indicating that the thermometer (3) has been removed from the case (4). As a further example, the process may include additionally or alternatively detecting that the user has pushed a button on a user interface of the thermometer, or otherwise provided input that the alarm should be turned off. As a further example, the process may include additionally or alternatively detecting that the thermometer (3) has taken a temperature, such as a temperature of the user.

The process may additionally include, in response to the user operation, operating a wireless transceiver (e.g., a BLE transceiver) of the thermometer (3) to transmit or advertise one or more messages to the smartphone or other device. The message(s) may include any suitable content, as embodiments are not limited in this respect. In some embodiments, the message(s) may indicate that a user has picked up and/or begun operating the thermometer. In other embodiments, the message(s) may include an indication that the user appears to be awake. In still other embodiments, the message(s) may include a “turn alarm off” instruction, or other suitable instruction, to the smartphone or other device. The “turn alarm off” message may be any suitable message according to any suitable protocol, as embodiments are not limited in this respect.

Once the smartphone or other device receives the message via its own wireless transceiver (e.g., another BLE transceiver), the message may be processed by any suitable software process on the smartphone/device. For example, in some embodiments, the message may be presented to a fertility data collection and/or charting application on the smartphone, which may in response communicate to an alarm application, fertility charting application, phone operating system, or other process to turn off the alarm.

Embodiments have been described in which an alarm is audibly, haptically, and/or visually output on a smartphone (or other device) and a thermometer transmits one or more messages to the smartphone to cease outputting of the alarm. In some embodiments, the thermometer (3) may also include a user interface to output an alarm. For example, an alarm may be output from a thermometer (3) via one or more speakers of the thermometer (3), one or more displays of the thermometer (3), and/or a vibration module of the thermometer (3), and/or via one or more displays, speakers, or vibration modules of case (4). In some such embodiments, the smartphone may include a software facility (which may be integrated with a fertility charting application, as a part of any other suitable application or operating system, or as a standalone facility) that, in response to detecting that the smartphone is outputting an alarm or that a condition is met for the smartphone to output an alarm, transmits (e.g., wirelessly) one or more messages to a thermometer (3) and/or case (4). The thermometer (3) and/or case (4) may be configured (e.g., via a programmed controller) to, in response to the message(s), output an alarm from the thermometer (3) and/or case (4). The thermometer (3) and/or case (4), in some such embodiments, may be additionally configured to cease an outputting of the alarm in response to the same stimuli discussed above in this section, including a user picking up the thermometer (3) and/or case (4), changing a state of a switch, operating the thermometer (3) to take a temperature, etc. Accordingly, in response to one or more of such stimuli, in some embodiments the thermometer (3) may cease outputting an alarm as well as transmit an instruction to a smartphone or other device to cease outputting an alarm. In such embodiments, the smartphone or other device may or may not output the alarm at a time when the thermometer (3) and/or case (4) are outputting the alarm. For example, in some embodiments the smartphone may detect that a condition is met to output an alarm and, in response, transmit an instruction to the thermometer (3) and/or case (4) to output the alarm, but the smartphone/device may not output an alarm. In some such embodiments, the smartphone/device may periodically send another instruction to the thermometer (3) and/or case (4) to output the alarm again, unless and/or until the smartphone/device receives from the thermometer (3) and/or case (4) an instruction to cease outputting the alarm.

In some embodiments in which a thermometer (3) and/or case (4) are configured to output an alarm, the thermometer (3) and/or case (4) may be configured to receive a configuration instruction including a date and/or time at which an alarm is to be output, and to output an alarm in response to determining that a current time matches the date and/or time of the configuration instruction. The configuration instruction may be received via a user interface of the thermometer (3) and/or case (4). Alternatively, the configuration instruction may be included in one or more messages received (e.g., wirelessly) from a smartphone or other device. In some embodiments in which the configuration instruction is received wirelessly from the smartphone or other device, the smartphone may transmit the message(s) directly to the thermometer (3) and/or case (4), such as following an initialization in which the smartphone is paired with the thermometer (3) and/or case (4). In other embodiments, the smartphone may continuously, periodically, and/or occasionally broadcast (e.g., every 10 seconds for one second) the message(s) including the configuration instruction and the thermometer (3) and/or case (4) may continuously, periodically, and/or occasionally scan (e.g., every 0.5 seconds for 0.1 seconds) for such broadcasted messages. In response to receiving the broadcasted message, the thermometer (3) and/or case (4) may configure itself to output an alarm when a current time matches the date and/or time of the configuration instruction. In some such embodiments, the thermometer (3) and/or case (4) may initiate the continuous, periodic, or occasional scanning for broadcasted messages in response to a user input, such as an input provided via a user interface of the thermometer (3) and/or case (4) (e.g., a button) that instructs the scanning.

Some embodiments in which a thermometer (3) and/or case (4) are configured to receive such configuration instructions and output an alarm may be advantageous in that these embodiments may permit a smartphone or other device to be turned off and do not require the smartphone or other device to be powered on when the alarm is to be output, because the alarm may be output via the thermometer (3) and/or case (4).

Ninth Functionality

With reference to FIG. 2C, in some embodiments, the thermometer (3) does not have a separate, specific “on/off” button. In these embodiments, the thermometer (3) may instead uses one or both of an accelerometer and a position sensor to detect that it should be “on” or “off.”

In some embodiments, this functionality may be implemented by a processor of the circuit board (6) and/or circuit board (17) executing instructions stored in one or more storages. The instructions may embody a process including various acts.

The process may include detecting a user operation, including any of the user operations discussed above in connection with the section titled “Eighth Functionality.” For example, detection of the user operation may be carried out by detecting a signal from one or more accelerometers of the thermometer (3). The process may also include, in response to the user operation, turning the thermometer (3) “on.” Turning the thermometer on may include any suitable operations, as embodiments are not limited in this respect. For example, in some embodiments, turning the thermometer (3) on may include illuminating the screen (8) and displaying any suitable information, and/or initializing the control circuit (6) to take a temperature measurement.

This feature may save a step for the user (i.e., a step to specifically turn on the device), since in normal use the thermometer will turn on when it is taken out of its case. This feature may also increase the device's portability and battery life, since the device will remain off as long as the thermometer (3) remains in the case (4).

Tenth Functionality

With reference to FIGS. 2A-2C, in some embodiments, the probe guide (13) inside the case includes a neck (57), which is a cavity into which the probe (9) and probe (10) can be inserted, that is shaped to ensure the thermometer is inserted correctly into the case. If the thermometer is inserted into the case such that (using the x and y axes as illustrated in FIG. 2A) the x axis of the thermometer is parallel to the y axis of the case, the guide neck is shaped such that it will exert a rotational force on the probe tip. This rotational force is one that influences the thermometer to rotate up to 90 degrees, in order that the x axes of the thermometer and of the case align, ensuring proper mating of the two parts. This may be accomplished by shaping the guide neck (57) such that it is slightly thinner (e.g., 70 to 90 percent of the width of) than the probe tip in the x dimension, but wider than (e.g., 110 to 130 percent wider than) the probe tip in the y dimension. In the case that the user inserts the thermometer into the case in the incorrect orientation, this geometry enables a translation of the force exerted by the user in mating the two parts into a rotational force that influences a proper alignment of the thermometer (3) and the case (4).

### Exemplary Embodiments

Techniques operating according to the principles described herein may be implemented in any suitable manner. In some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as one or more computer-readable storage media of a computing device or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

1-15. (canceled)

16. An apparatus comprising:
a thermometer, the thermometer comprising
a temperature sensor;
at least one processor; and
at least one storage having encoded thereon executable instructions that, when executed by the at least one processor, cause the at least one processor to carry out a method comprising:
following a start of reading of a temperature with the temperature sensor, detecting a completion of the reading of the temperature; and
in response to detecting the completion, providing a signal to a user that the reading is complete, wherein providing the signal comprises providing a haptic and/or visual signal to the user.
